(19) United States
(12) Patent Application Publication
(10) Pub. No.: US 2015/0014432 A1
(43) Pub. Date: Jan. 15, 2015

(54) Fragrance Sticks

(71) Applicant: Audrey Kieschnick, Virginia Beach, VA (US)

(72) Inventor: Audrey Kieschnick, Virginia Beach, VA (US)

(21) Appl. No.: 13/937,287

(22) Filed: Jul. 9, 2013

(51) Int. Cl. A61L 9/12

(52) U.S. Cl. CPC A61L 9/12
USPC 239/34

(57) ABSTRACT

A device for displaying fragrance strips. The device includes a container with slots and one or more fragrance strips that fit into the slots. The fragrance strips can be constructed with aesthetically pleasing or branding and marketing designs. The container can also be constructed with aesthetically pleasing or branding and marketing designs. The container may also be collapsible.

### BACKGROUND OF THE INVENTION

The present invention relates to perfume and perfume-related accessories.

It is common practice for sellers and marketers of perfumes and colognes to apply the fragrance on paper so that a customer can smell the fragrance and determine whether he or she would like to use and/or purchase the perfume or cologne. Other types of fragrant sticks and the like are also used to allow a fragrance in the air of a room.

When using devices, whether fragrance sticks or not, to perfume the air of a room, it is desirable to have devices that are attractive as well as functional. It is also desirable to have devices that appeal to a wide range of individuals. It is also desirable to have fragrance devices that are easy to use and will provide a user with easy and attractive means to fragrance a room with their favorite cologne or perfume. It is also desirable to have a cost-effective yet attractive and useful means of providing samples of perfumes and colognes to purchasers and potential purchasers.

### SUMMARY OF THE INVENTION

The present invention is directed to a new and improved invention related to the sale and marketing of perfumes and colognes.

It is an object of the present invention to provide for a new, improved device for providing samples of perfumes and colognes.

It is a further object of the present invention to provide a new, improved device for providing samples of perfumes and colognes, the device being are attractive as well as functional.

It is a further object of the present invention to provide an improved device for providing room and other location fragrance enhancement.

It is a further object of the present invention to provide an improved device for providing samples of perfumes and colognes that appeal to a wide range of individuals.

It is a further object to provide a cost-effective yet attractive and useful means of providing samples of perfumes and colognes to purchasers and potential purchasers.

### DESCRIPTION OF THE DRAWINGS

FIG. 1 is a view of an embodiment of the present invention showing a display device.

FIG. 2, 2(a)-2(e), are views of embodiments of the present invention showing various forms and shapes to be used with fragrance sticks in accordance with the present invention.

FIG. 3 is a view of an embodiment of the present invention showing the invention with a fragrance stick retained within a display device.

FIG. 4 is a view of an embodiment of the present invention showing a display device with multiple slots.

### DESCRIPTION OF A PREFERRED EMBODIMENT

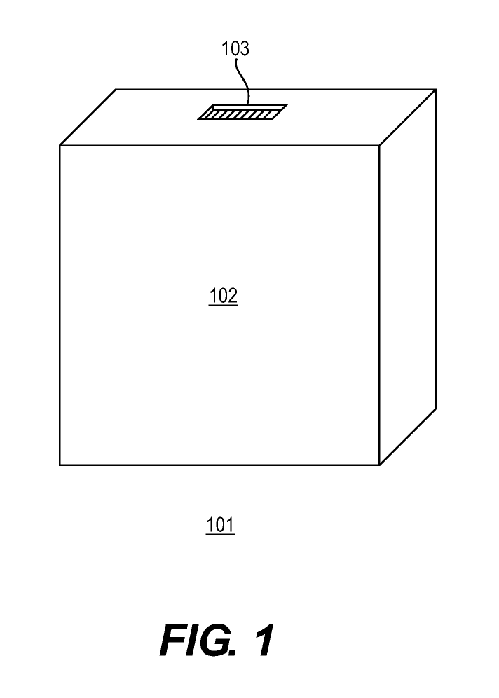

Referring now to FIG. 1, an embodiment of the current invention is shown. FIG. 1 shows a container 101 in accordance with an embodiment of the present invention. The container body 102 is shown as a cube with three sides visible. It will be understood that the container body, when positioned on a surface, will of necessity have a top portion and a bottom portion, regardless of the container body shape chosen. The container body 102 further comprises an opening, referred to herein as a slot 103 which is shown positioned on the top portion of the container body, but it will be understood that the slot may be positioned in other aesthetically-pleasing locations on the container body 102. The present invention may also contain multiple slots without deviating from the scope of the invention.

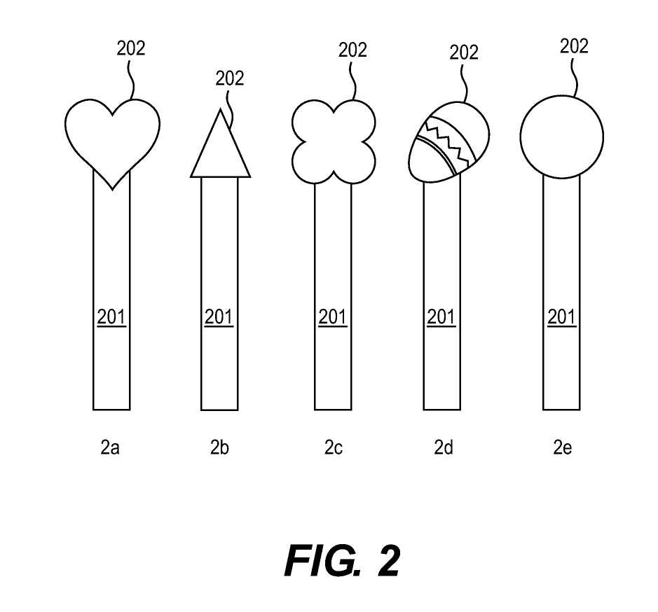

The slot 103 is configured to accommodate a strip (FIG. 2, 201). While the container body 102 is shown as a cube, it will be understood that there are various shapes and sizes that the container body 102 may take; any regular or irregular shape may be used, including shapes that incorporate branding and/or marketing messages, logos, symbols, and the like. It will also be understood that the container body 102 may be foldable and/or collapsible, such that the container body 102 in folded or collapsed state may be provided to a customer or potential customer. It will also be understood that the container body may be used, in configuration and/or decoration, to function as marketing material, such as including the color scheme and/or name of the fragrance with which it is associated.

Referring now to FIG. 2, fragrance sticks 201 are shown in accordance with an embodiment of the present invention. The fragrance sticks 201 may be constructed from various porous and/or commonly known materials that either absorb cologne or perfume, or to which cologne or perfume will adhere. The perfume industry commonly uses paper products because of the absorption properties and the low cost of such material. However, other materials may be used, such as woods and the like. Affixed to the top portion of the fragrance sticks 201 are larger absorption areas 202. As shown (FIGS. 2(a)-(e)), various decorative or other shapes may be utilized for the absorption areas 202. The absorption areas 202 serve to provide greater retention of the applied cologne or perfume, but may also be used for decorative purposes. For example, shapes may be decided by holidays (the heart on FIG. 2(a), the four leaf clover on FIG. 2(c), and the Easter egg on FIG. 2(d)) or by other determining factors such as using the logos associated with the products, denominational or non-denominational religious symbols, letters used to correspond to the name of a potential customer, and the like.

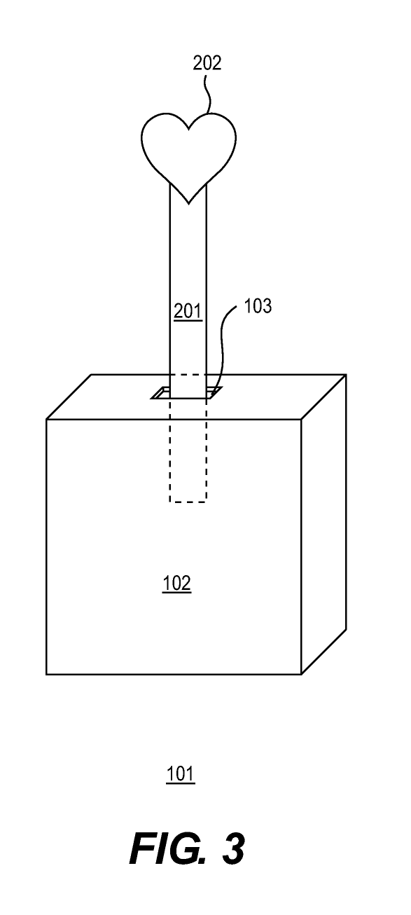

Referring now to FIG. 3, the container 101 is shown with the container body 102 and the slot 103, with a fragrance stick 201 inserted into the container body, with an absorption area 202 on the top of the fragrance stick 201 above the container body 102. The invention as shown does not have the fragrance stick 201 shown inserted completely into the container 101. It will be understood that the size and shape of the container 101 and the fragrance stick 201 may be modified to accommodate different needs for marketing and the like, and the length of the fragrance stick 201 as well as the depth and shape of the container 101 may vary accordingly depending upon the particular need or desire without deviating from the scope of this invention.

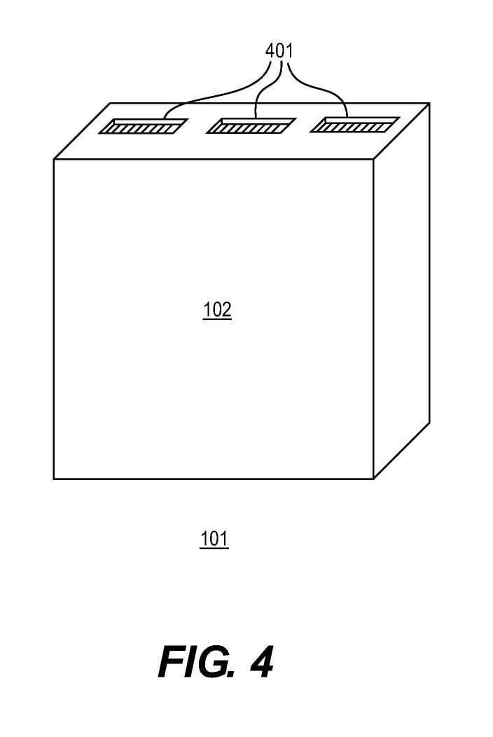

Referring now to FIG. 4, an embodiment of the current invention is shown. The container body 102 is shown as a cube with three sides visible. The container body 102 further comprises multiple slots 401. The slots 401 are configured to accommodate a strip (FIG. 2, 201).

When in use, a fragrance is applied to the fragrance stick 201 and/or the absorption area 202. The combination fragrance stick 201, inserted into the container 101, may then be positioned in a room as desired. The combination may also be displayed at fragrance counters and the like in order to accommodate marketing of the perfume and/or cologne to be associated with the invention, and the shape of the container may be chosen in a manner intended to serve as marketing and/or branding for the perfume and/or cologne sought to be displayed.

It will be understood that, while the invention is described in detail herein, embodiments exist which are not described herein but which are covered by and fall within the spirit and the scope of the invention as described.

What is claimed:

1. A container, the container consisting of an exterior portion and a hollow interior portion, the exterior portion having one or more sides, a top portion and a bottom portion, the top portion further having one or more openings into the hollow interior portion.

2. The container of claim 1, the container further comprising one or more removable strips, the one or more removable strips having a portion with a width no greater than the width of the one or more openings and a length sufficient to allow the one or more strips, when inserted fully into the one or more openings, to extend across the hollow interior and rest against an interior surface opposite to the one or more openings.

3. The container of claim 2 wherein the one or more removable strips are formed from a material that will absorb fragrant liquids.

4. The container of claim 2 wherein the one or more removable strips further comprise a larger surface area at one end of the removable strip.

5. The container of claim 4 wherein the larger surface area comprises visually pleasing shapes.

6. The container of claim 2 wherein the container is shaped in a visually pleasing manner.

7. The container of claim 2 wherein the container is shaped in a manner to convey branding and marketing information related to a fragrance product.

8. A container, the container consisting of
an exterior portion and a hollow interior portion, the exterior portion having one or more sides;
a top portion and a bottom portion, the top portion further having one or more openings into the hollow interior portion;
the container further comprising one or more removable strips, the one or more removable strips having a portion with a width no greater than the width of the one or more openings and a length sufficient to allow the one or more strips, when inserted fully into the one or more openings, to extend across the hollow interior and rest against an interior surface opposite to the one or more openings with a portion of the removable strip disposed outside of the container;
the one or more removable strips are formed from a material that will absorb fragrant liquids;
the one or more removable strips further comprise a larger surface area at one end of the removable strip; and
the larger surface area are visually pleasing shapes.
